(19) United States
(12) Patent Application Publication
(10) Pub. No.: US 2022/0409590 A1
(43) Pub. Date: Dec. 29, 2022

(54) CHEMOTHERAPY FOR GLIOMA THROUGH NEURONAL CONVERSION

(71) Applicant: The Penn State Research Foundation, University Park, PA (US)

(72) Inventors: Gong CHEN, Guangzhou (CN); Ivan SOKIRNIY, State College, PA (US)

(73) Assignee: The Penn State Research Foundation, University Park, PA (US)

(21) Appl. No.: 17/779,055

(22) PCT Filed: Nov. 25, 2020
(86) PCT No.: PCT/US2020/062247

§ 371 Date: May 23, 2022

Related U.S. Application Data
(60) Provisional application No. 62/940,000, filed on Nov. 25, 2019.

(51) Int. Cl. A61K 31/422; A61K 31/4439; A61K 31/519; A61K 31/506; A61K 31/616; A61P 35/00

(52) U.S. Cl. CPC A61K 31/422; A61K 31/4439; A61K 31/519; A61K 31/506; A61K 31/616; A61P 35/00

(57) ABSTRACT

Compositions and methods for treating glioma and converting glioma cells into neurons are provided. The compositions contain an effective amount of SKL2001, and may also contain one, two, three or four of 0431542, LDN193189, CHIR99021, and DAPT. Pharmaceutical compositions containing SKL2001 and one, two, three or four of SB431542, LDN193189, CHIR99021, and DAPT are provided. Also provided are articles of manufacture which contain one or more sealed containers that contain SKL2001 and one, two, three or four of SB431542, LDN193189, CHIR99021, and DAPT, are provided. The articles of manufacture can contain printed material providing an indication that the compound(s) are for use in treating glioma.

### CROSS REFERENCE TO RELATED APPLICATIONS

This application claims priority to U.S. Provisional Patent Application No. 62/940,000, filed Nov. 25, 2019, the entire disclosure of which is incorporated herein by reference.

### FIELD

The present disclosure relates generally to compositions and methods for treating glioma. The compositions comprise a compound known as SKL2001, alone or in combination with one or more additional agents that are SB431542, LDN193189, CHIR99021, and DAPT. Combining SKL2001 with the one or more additional agents improves the efficiency of the conversion of the glioma cells to neuronal cells.

### BACKGROUND

Given the significant advances in cancer treatment, the overall 5-year survival of rates of glioblastoma multiforme (GBM) has stagnated just below 5%.¹ The current standard of care for treating newly diagnosed GBM include resection, radiation, and treatment with the DNA alkylating agent temozolomide.^{2,3} Additionally, glioma stem cells (GSC) have been implicated in GBM chemoresistance, heterogeneity, and reoccurance.^{4,5} While temozolomide has been shown to target GSC⁶, it can also potentially make them more tumorigenic.⁷ Thus, a therapeutic strategy to combat GBM is needed. The present disclosure is applicable to this need.

### SUMMARY

Compositions and methods for converting malignant glial cells (e.g., glioma) into neurons are provided. The disclosure demonstrates that a compound known in the art as SKL2001 provides efficient conversion of glioma cells to neuronal cells. Combining SKL2001 with one or more of SB431542, LDN193189, CHIR99021, or DAPT improves the efficiency of conversion of glioma cells to neurons. The method is therefore suitable for use with SKL2001 alone, or with SKL2001 and any one or more of SB431542, LDN193189, CHIR99021, and DAPT.

In an embodiment the disclosure thus provides a method for converting glioma cells into neurons, the method comprising contacting the glioma cells with an effective amount of SKL2001.

In an embodiment, the disclosure provides a pharmaceutical composition comprising a combination of SKL2001 and at least one of SB431542, LDN193189, CHIR99021, or DAPT.

In an embodiment, the disclosure provides an article of manufacture comprising a pharmaceutical composition comprising SKL2001 and at least one of SB431542, LDN193189, CHIR99021, or DAPT, the article of manufacture further comprising printed material providing an indication that the pharmaceutical composition is for use in treating a glioma.

### BRIEF DESCRIPTION OF THE FIGURES

FIG. 1. Immunohistochemistry images showing SKL2001 in combination with SB431542, LDN193189, CHIR99021, and DAPT (SLCD+SKL2001) dramatically enhances doublecortin (DCX) expression in U251 cells.

FIG. 2. Immunohistochemistry images showing dose response of SKL2001 in combination with SB431542, LDN193189, CHIR99021, and DAPT in U251 cells.

FIG. 3. Structure of SB431542.

FIG. 4. Structure of LDN193189.

FIG. 5. Structure of CHIR99021

FIG. 6. Structure of DAPT.

FIG. 7. Structure of SKL2001.

### DETAILED DESCRIPTION

Unless defined otherwise herein, all technical and scientific terms used in this disclosure have the same meaning as commonly understood by one of ordinary skill in the art to which this disclosure pertains.

When a grouping of alternatives is presented, any and all combinations of the members that make up that grouping of alternatives is specifically envisioned. For example, if an item is selected from a group consisting of A, B, C, and D, the inventors specifically envision each alternative subjectly (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

Every numerical range given throughout this specification includes its upper and lower values, as well as every narrower numerical range that falls within it, as if such narrower numerical ranges were all expressly written herein. When a range of numbers is provided herein, the range is understood to be inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

The singular terms “a” “an” and “the” are not intended to be limiting and include plural referents unless explicitly stated otherwise or the context clearly indicates otherwise.

Any result obtained using a method or composition described herein can be compared to any suitable reference, such as a known value, or a control sample or control value, suitable examples of which will be apparent to those skilled in the art, given the benefit of this disclosure.

The present disclosure comprises compositions and methods that are designed to convert glioma cells into neurons. In this regard, the disclosure includes data demonstrating that contacting U251 cells with SKL2001 provides efficient conversion of U251 glioblastoma cells to neuronal cells, as evidenced by doublecortin expression. The data also demonstrate that combining SKL2001 with SB431542, LDN193189, CHIR99021, and DAPT improves the efficiency of conversion of U251 glioblastoma cells to neurons. The latter four compounds are referred to herein from time to time as “SLCD.” A five compound combination that includes the latter four compounds and SKL2001 is referred to herein from time to time as “SLCDS.” Each of these SLCD compounds is known in the art and is commercially available. Representative structures of these compounds are presented in the figures. The disclosure includes pharmaceutically acceptable salts of each of the described compounds.

U251 cells, which are also referred to in the art as U-251 cells, and were formerly known as U-373 cells, are commercially available, such as from MILLIPORE-SIGMA as product no. 09063001. The cells are derived from a malignant glioblastoma tumor, and in particular, a human glioblastoma astrocytoma. Thus, while certain embodiments of the disclosure are demonstrated in vitro, it is expected the data can be extended to in vivo applications, including but not limited to animal models of glioma and related brain cancers, and for use in treating humans. The disclosure includes prophylactic and therapeutic approaches using the described compound combinations.

All combinations of SKL2001 with one or more of SB431542, LDN193189, CHIR99021, and DAPT are encompassed by the disclosure. In embodiments, SKL2001 may be the only compound that is used to contact glioma cells and convert them into neurons. In embodiments, the SKL2001 compound is used in combination with at least one of SB431542, LDN193189, CHIR99021, and DAPT. In non-limiting embodiments, the disclosure thus provides compositions, methods, and articles of manufacture that include:

SKL2001 and SB431542;

SKL2001 and LDN193189;

SKL2001 and CHIR99021;

SKL2001 and DAPT;

SKL2001 with SB431542 and LDN193189;

SKL2001 with SB431542 and CHIR99021;

SKL2001 with SB431542 and DAPT;

SKL2001 with LDN193189, CHIR99021;

SKL2001 with CHIR99021, and DAPT;

SKL2001 with SB431542, LDN193189 and CHIR99021;

SKL2001 with LDN193189, CHIR99021, and DAPT;

SKL2001 with SB431542, CHIR99021, and DAPT; and

SKL2001 with SB431542, LDN193189, CHIR99021, and DAPT.

The disclosure includes compositions and methods that comprise or consist of SKL2001 or use of SKL2001 alone, and any of the foregoing compound combinations that include SKL2001.

In embodiments, contacting glioma cells with a described compound (e.g. SKL2001) or a described combination of compounds comprises administering the compound or combination of compounds to a subject in need thereof. Thus, the disclosure includes in a non-limiting embodiment administering to a subject in need thereof SKL2001 alone, or in any of the described combinations that include SKL2001.

In embodiments, the subject in need of a described method has a CNS malignancy (e.g., a cancer), including but not necessarily limited to a brain or brain stem disorder. In embodiments, the cancer comprises only a single type of glioma. In embodiments, the cancer comprises more than one type of glioma cell, e.g., a mixed glioma. In embodiments, the glioma is a high grade glioma, or a low grade glioma. Staging of glioma into grades is well known in the art. In general, low grade gliomas are considered to be Stage I or Stage II. A non-limiting embodiment of a Stage I glioma is juvenile pilocytic astrocytoma. Grade I gliomas may have mutations in the human BRAF gene. The most common Grade II gliomas are astrocytomas and oligodendrogliomas. Grade II gliomas frequent have mutations in the human IDH1 or IDH2 gene. High-grade gliomas are grade III or grade IV tumors. Grade III gliomas include anaplastic astrocytomas and anaplastic oligodendrogliomas. Grade IV gliomas are referred to a glioblastomas. It is considered, without intending to be bound by any particular theory, that the described compounds and methods are suitable for treating any of the cancer that comprises one or more of the described types of gliomas. In embodiments, the cancer thus comprises one or more astrocytomas, which include but are not necessarily limited to astrocytoma, anaplastic astrocytoma and glioblastoma. In embodiments, the cancer comprises an ependymoma, including but not necessarily limited to anaplastic ependymoma, myxopapillary ependymoma and subependymoma. In embodiments, the cancer comprises an oligodendroglioma, including but not limited to oligodendroglioma, anaplastic oligodendroglioma and anaplastic oligoastrocytom. In embodiments, the cancer comprises a low-grade oligodendroglioma. In embodiments, the cancer comprises optic nerve glioma. In embodiments, the cancer comprises a subependymoma. In embodiments, the cancer comprises a brain stem glioma. In embodiments, the described approach reduces or eliminates glioma stem cells. In embodiments, the described approach is therapeutic for glioblastoma. In embodiments , the described approach is therapeutic for glioblastoma multiforme.

Contacting glioma cells with the described compound (e.g., SKL2001 alone), or SKL2001 with one or more of SB431542, LDN193189, CHIR99021, and DAPT, is expected to result in at least some glioma cells in the subject being converted into neurons. In embodiments, conversion into neurons takes place over a period of approximately 7 to 14 days. In embodiments, conversion into neurons takes place between 7 days and 8 days, between 7 days and 9 days, between 7 days and 10 days, between 8 days and 9 days, between 8 days and 10 days, between 8 days and 11 days, between 9 days and 10 days, between 9 days and 11 days, between 9 days and 12 days, between 10 days and 11 days, between 10 days and 12 days, between 10 days and 13 days, between 11 days and 12 days, between 11 days and 13 days, between 11 days and 14 days, between 12 days and 13 days, between 12 days and 14 days, or between 13 days and 14 days. In embodiments, conversion into neurons does not take more than 7, 8, 9, 10, 11, 12, 13, 14, 15, 16, 17, 18, 19, 20, or 21 days.

In addition to converting glioma cells into neurons, the disclosure further comprises generation of new neurons from endogenous glial cells, and can include generating new neurons from glia-like cells created due to injury or a disease condition (such as but not limited to glioma) in the central or peripheral nervous system.

The neurons generated using the described compositions and methods are generally functional neurons, and thus are capable of at least one of the following non-limiting example functions: synapse formation, axon formation, dendrite formation, or neurotransmitter release. Non-limiting examples of the generated neurons produced by the described methods and compositions are unipolar, bipolar, or multipolar neurons. Non-limiting examples of the neurons comprise any type of neuron, such as basket cells, Lugaro cells, medium spiny neurons, Purkinje cells, or spindle cells. In embodiments, the neurons are selected from the group consisting of cholinergic neurons, GABAergic neurons, glutamatergic neurons, dopaminergic neurons, epinephrinergic neurons, motor neurons, peptidergic neurons, and serotonergic neurons.

Functional neurons can exhibit properties which can comprise but are not necessarily limited to firing repetitive action potentials, developing a plurality of dendritic branches, and release of neurotransmitters, including but not necessarily limited to Glutamate (glutamic acid), dopamine, acetylcholine, serotonin, Norepinephrine (noradrenaline), and γ-Aminobutyric acid (GABA). Thus, the disclosure is expected to facilitate development of new cortical forebrain neurons, or midbrain neurons, or hindbrain neurons, or spinal cord neurons, or peripheral neurons, or combinations thereof by using methods described herein adapted as necessary by those skilled in the art in a manner that will be apparent given the benefit of the present disclosure. In embodiments, glioma cells are converted into neurons that express one or both of the neuronal markers NeuN (also referred to as NEUN) or Microtubule-associated protein 2 (MAP-2). In embodiments, the generated neurons express doublecortin (DCX). In embodiments, the generated neurons express reduced levels of glial fibrillary acidic protein (GFAP). In embodiments, the generated neurons do not express GFAP. In embodiments, the neurons are post-mitotic neurons. In embodiments, the generated neurons comprise post-mitotic neurons that expressDCX. In this regard, while there have been previous attempts to target glioma using various drug cocktails^{8,9}, and without intending to be bound by any particular scientific theory, it is considered that no previous methods have targeted a glioma stem cell (GSC) population of glioblastoma. In connection with this, activation of the WNT/β-catenin pathway has been specifically shown to be implicated in neuronal differentiation of the GSC subpopulation of GBM cells.¹⁰ The compound SKL2001 rescues β-catenin from degradation by destabilizing the Axin/β-catenin protein complex, and ultimately inducing differentiation of mesenchymal stem cells.¹² Data presented in this disclosure demonstrates that combining SKL2001 with SLCD promotes appearance of DCX, an early neuronal marker, in the U251 glioblastoma cell line. DCX is known in the art, and is also referred to as neuronal migration protein doublecortin, doubling, lissencephalin-X.

As used herein, the term “subject” refers to any animal subject. Non-limiting examples of animal subjects include humans, laboratory animals (e.g., non-human primates, rats, mice), livestock (e.g., cows, sheep, goats, pigs, turkeys, chickens), and household pets (e.g., dogs, cats, rodents, etc.).

In embodiments, a subject is in need of neuronal generation. In embodiments, the subject in need of neuronal generation has glioma.

In embodiments, a subject in need thereof is a male. In embodiments, a subject in need thereof is a female. In embodiments, a subject in need thereof is gender neutral. In embodiments, a subject in need thereof is a premature newborn. In embodiments, a premature newborn is born before 36 weeks gestation. In embodiments, a subject in need thereof is a term newborn. In embodiments, a term newborn is below about 2 months old. In embodiments, a subject in need thereof is a neonate. In embodiments, a neonate is below about 1 month old. In embodiments, a subject in need thereof is an infant. In embodiments, an infant is between 2 months and 24 months old. In embodiments, an infant is between 2 months and 3 months, between 2 months and 4 months, between 2 months and 5 months, between 3 months and 4 months, between 3 months and 5 months, between 3 months and 6 months, between 4 months and 5 months, between 4 months and 6 months, between 4 months and 7 months, between 5 months and 6 months, between 5 months and 7 months, between 5 months and 8 months, between 6 months and 7 months, between 6 months and 8 months, between 6 months and 9 months, between 7 months and 8 months, between 7 months and 9 months, between 7 months and 10 months, between 8 months and 9 months, between 8 months and 10 months, between 8 months and 11 months, between 9 months and 10 months, between 9 months and 11 months, between 9 months and 12 months, between 10 months and 11 months, between 10 months and 12 months, between 10 months and 13 months, between 11 months and 12 months, between 11 months and 13 months, between 11 months and 14 months, between 12 months and 13 months, between 12 months and 14 months, between 12 months and 15 months, between 13 months and 14 months, between 13 months and 15 months, between 13 months and 16 months, between 14 months and 15 months, between 14 months and 16 months, between 14 months and 17 months, between 15 months and 16 months, between 15 months and 17 months, between 15 months and 18 months, between 16 months and 17 months, between 16 months and 18 months, between 16 months and 19 months, between 17 months and 18 months, between 17 months and 19 months, between 17 months and 20 months, between 18 months and 19 months, between 18 months and 20 months, between 18 months and 21 months, between 19 months and 20 months, between 19 months and 21 months, between 19 months and 22 months, between 20 months and 21 months, between 20 months and 22 months, between 20 months and 23 months, between 21 months and 22 months, between 21 months and 23 months, between 21 months and 24 months, between 22 months and 23 months, between 22 months and 24 months, and between 23 months and 24 months old. In embodiments, a subject in need thereof is a toddler. In embodiments, a toddler is between 1 year and 4 years old. In embodiments, a toddler is between 1 year and 2 years, between 1 year and 3 years, between 1 year and 4 years, between 2 years and 3 years, between 2 years and 4 years, and between 3 years and 4 years old. In embodiments, a subject in need thereof is a young child. In embodiments, a young child is between 2 years and 5 years old. In embodiments, a young child is between 2 years and 3 years, between 2 years and 4 years, between 2 years and 5 years, between 3 years and 4 years, between 3 years and 5 years, and between 4 years and 5 years old. In embodiments, a subject in need thereof is a child. In embodiments, a child is between 6 years and 12 years old. In embodiments, a child is between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, and between 11 years and 12 years old. In embodiments, a subject in need thereof is an adolescent. In embodiments, an adolescent is between 13 years and 19 years old. In embodiments, an adolescent is between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, between 16 years and 19 years, between 17 years and 18 years, between 17 years and 19 years, and between 18 years and 19 years old. In embodiments, a subject in need thereof is a pediatric subject. In embodiments, a pediatric subject between 1 day and 18 years old. In embodiments, a pediatric subject is between 1 day and 1 year, between 1 day and 2 years, between 1 day and 3 years, between 1 year and 2 years, between 1 year and 3 years, between 1 year and 4 years, between 2 years and 3 years, between 2 years and 4 years, between 2 years and 5 years, between 3 years and 4 years, between 3 years and 5 years, between 3 years and 6 years, between 4 years and 5 years, between 4 years and 6 years, between 4 years and 7 years, between 5 years and 6 years, between 5 years and 7 years, between 5 years and 8 years, between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, between 10 years and 13 years, between 11 years and 12 years, between 11 years and 13 years, between 11 years and 14 years, between 12 years and 13 years, between 12 years and 14 years, between 12 years and 15 years, between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, and between 17 years and 18 years old. In embodiments, a subject in need thereof is a geriatric subject. In embodiments, a geriatric subject is between 65 years and 95 or more years old. In embodiments, a geriatric subject is between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In embodiments, a subject in need thereof is an adult. In embodiments, an adult subject is between 20 years and 95 or more years old. In embodiments, an adult subject is between 20 years and 25 years, between 20 years and 30 years, between 20 years and 35 years, between 25 years and 30 years, between 25 years and 35 years, between 25 years and 40 years, between 30 years and 35 years, between 30 years and 40 years, between 30 years and 45 years, between 35 years and 40 years, between 35 years and 45 years, between 35 years and 50 years, between 40 years and 45 years, between 40 years and 50 years, between 40 years and 55 years, between 45 years and 50 years, between 45 years and 55 years, between 45 years and 60 years, between 50 years and 55 years, between 50 years and 60 years, between 50 years and 65 years, between 55 years and 60 years, between 55 years and 65 years, between 55 years and 70 years, between 60 years and 65 years, between 60 years and 70 years, between 60 years and 75 years, between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In embodiments, a subject in need thereof is between 1 year and 5 years, between 2 years and 10 years, between 3 years and 18 years, between 21 years and 50 years, between 21 years and 40 years, between 21 years and 30 years, between 50 years and 90 years, between 60 years and 90 years, between 70 years and 90 years, between 60 years and 80 years, or between 65 years and 75 years old. In embodiments, a subject in need thereof is a young old subject (65 to 74 years old). In embodiments, a subject in need thereof is a middle old subject (75 to 84 years old). In embodiments, a subject in need thereof is an old subject (>85 years old).

In general, methods of the disclosure comprise administering an effective amount of the compounds described herein to a subject such that the number of neurons in the subject is increased. The compounds can be administered in amounts that are the same or similar to those for which FDA approval is already in place. Dosages for each of the FDA approved drugs can be found, for example, in www.accessdata.fda.gov/scripts/cder/drugsatfda/, the disclosure of which that pertains to the described compounds is incorporated herein by reference it exists on the effective filing date of this application or patent. Thus, appropriate dosing of the compound(s) can be determined in conjunction with the knowledge of the skilled artisan, given the benefit of the present disclosure. In embodiments, the weight and age of the subject, personal history of neuronal damage or disease and risk for experiencing same neuronal damage, or the presence of glial scarring or reactive gliosis, may be taken into account when determining an effective amount of the active ingredient and dosing regimen. In embodiments one or a combination of the described compounds are administered in an amount of about 0.01 nmol to about 500 nmol a day, inclusive, and including all integers and ranges there between, depending on which delivering method being used. In embodiments, the compounds are administered in an amount of about 0.01 nmol to about 25 nmol, about 0.01 nmol to about 50 nmol, about 0.01 nmol to about 75 nmol, about 25 nmol to about 50 nmol, about 25 nmol to about 75 nmol, about 25 nmol to about 100 nmol, about, about 50 nmol to about 75 nmol, about 50 nmol to about 100 nmol, about 50 nmol to about 125 nmol, about 75 nmol to about 100 nmol, to about 75 nmol to about 125 nmol, to about 75 nmol to about 150 nmol, to about 100 nmol to about 125 nmol to about 100 nmol to about 150 nmol, to about 100 nmol to about 175 nmol, 125 nmol to about 150 nmol, about 125 nmol to about 175 nmol, about 125 nmol to about 200 nmol, about 150 nmol to about 175 nmol, about 150 nmol to about 200 nmol, about 150 nmol to about 225 nmol, about 175 nmol to about 200 nmol, to about 175 nmol to about 225 nmol, to about 175 nmol to about 250 nmol, to about 200 nmol to about 225 nmol to about 200 nmol to about 250 nmol, to about 200 nmol to about 275 nmol, 225 nmol to about 250 nmol, about 225 nmol to about 275 nmol, about 225 nmol to about 300 nmol, about 250 nmol to about 275 nmol, about 250 nmol to about 300 nmol, about 250 nmol to about 325 nmol, about 275 nmol to about 300 nmol, to about 275 nmol to about 325 nmol, to about 275 nmol to about 350 nmol, to about 300 nmol to about 325 nmol to about 300 nmol to about 350 nmol, to about 300 nmol to about 375 nmol, 325 nmol to about 350 nmol, about 325 nmol to about 375 nmol, about 325 nmol to about 400 nmol, about 350 nmol to about 375 nmol, about 350 nmol to about 400 nmol, about 350 nmol to about 425 nmol, about 375 nmol to about 400 nmol, to about 375 nmol to about 425 nmol, to about 375 nmol to about 450 nmol, to about 400 nmol to about 325 nmol to about 400 nmol to about 450 nmol, to about 400 nmol to about 475 nmol, to about 425 nmol to about 450 nmol, to about 425 nmol to about 475 nmol, to about 425 nmol to about 500 nmol, to about 450 nmol to about 475 nmol, or to about 450 nmol to about 500 nmol day. In embodiments, one, or more than one of the compounds, are provided in a single, multiple, or controlled release dose regimen. In embodiments, such as where more than one compound is administered, the combination of compounds may be administered concurrently. In embodiments, more than one compound is administered sequentially. In embodiments, one or more of the compounds is/are administered as a component of the same pharmaceutical formulation. In embodiments, the only active ingredient(s) that is used to contact glioma cells and which may be in a pharmaceutical formulation comprises or consists of SKL2001. In embodiments, the only active ingredients in the combination is SKL2001, and at least one of SB431542, LDN193189, CHIR99021, or DAPT. In embodiments, an effective amount of the compound(s) is used. In embodiments, an effective amount (which may include a therapeutically acceptable amount) is an amount that can achieve a desired effect, such as reduction in glioma cells and/or glioma cells and/or glioma tumor growth rate, inhibition of glioma tumor formation, eradication of glioma cells, preventing a relapse of glioma, and/or inhibiting extension and/or infiltration into adjacent brain tissue along the white matter tract. In embodiments, extraneural metastasis of glioma is inhibited. In embodiments, an effective amount of the compound(s) is a value that is adjusted based at least in part on the concentration of compounds described in the example below, said value in embodiments reflecting a local concentration of the described compound(s) in the glioma cell environment.

“Active ingredient” means a compound that acts on the glioma cells to convert said cells to become neurons. Thus, “active ingredient” does not include agents that are added to, for example, a pharmaceutical formulation to contain or otherwise facilitate delivery of the active ingredients to the glial cells, such agents including but not necessarily limited to buffers, salts, pharmaceutically suitable excipients, carriers, and the like. Accordingly, in embodiments, the disclosure includes pharmaceutical formulations and methods of using said formulations wherein the only active ingredients in the pharmaceutical formulations consists of SKL2001, or consists of SKL2001 and one or more of SB431542, LDN193189, CHIR99021, or DAPT, as the compound combinations are described above, or otherwise herein.

In embodiments, the term “therapeutically effective dose,” “therapeutically effective amount,” “effective amount,” or “pharmaceutically active dose” refers to an amount of SB431542, LDN193189, CHIR99021, DAPT, and SKL2001, either alone or in combination that converts glioma cells to neurons. In embodiments, the therapeutically effective dose treats a glioma. In embodiments, an effective amount provides a concentration of the described compounds of 5μM to 8004, inclusive, and including all integers and ranges of integers there between. In embodiments, a therapeutically effective dose is provided as a concentration in the cell culture media of a cell culture. In embodiments, a therapeutically effective dose is provided as a concentration to a subject upon administration. In embodiments, a therapeutically effective dose is provided as a concentration in the blood of a subject upon administration. In embodiments, a therapeutically effective does is provided as a concentration in the brain of a subject upon administration. In embodiments, a therapeutically effective does is provided as a concentration in the spinal cord of a subject upon administration.

In embodiments, the therapeutically effective dose of SB431542 is between 1 μM and 20 μM. In embodiments, the therapeutically effective dose of SB431542 is between 1 μM and 5 μM, between 1 μM and 10 μM, between 1 μM and 15 μM, between 5 μM and 10 μM, between 5 μM and 15 μM, between 5 μM and 20 μM, between 10 μM and 15 μM, between 10 μM and 20 μM, or between 15 μM and 20 μM. In an embodiment, an effective amount provides a concentration of the described compounds of 5 μM to 80 μM, inclusive, and including all integers and ranges of integers there between.

In embodiments, the therapeutically effective dose of LDN193189 is between 0.1 μM and 1.0 μM. In embodiments, the therapeutically effective dose of LDN193189 is between 0.1 μM and 0.15 μM, between 0.1 μM and 0.2 μM, between 0.1 μM and 0.25 μM, between 0.15 μM and 0.2 μM, between 0.15 μM and 0.25 μM, between 0.15 μM and 0.3 μM, between 0.2 μM and 0.25 μM, between 0.2 μM and 0.3 μM, between 0.2 μM and 0.35 μM, between 0.25 μM and 0.3 μM, between 0.25 μM and 0.35 μM, between 0.25 μM and 0.4 μM, between 0.3 μM and 0.35 μM, between 0.3 μM and 0.4 μM, between 0.3 μM and 0.45 μM, between 0.35 μM and 0.4 μM, between 0.35 μM and 0.45 μM, between 0.35 μM and 0.5 μM, between 0.4 μM and 0.45 μM, between 0.4 μM and 0.5 μM, between 0.4 μM and 0.55 μM, between 0.45 μM and 0.5 μM, between 0.45 μM and 0.55 μM, between 0.45 μM and 0.6 μM, between 0.5 μM and 0.55 μM, between 0.5 μM and 0.6 μM, between 0.5 μM and 0.65 μM, between 0.55 μM and 0.6 μM, between 0.55 μM and 0.65 μM, between 0.55 μM and 0.7 μM, between 0.6 μM and 0.65 μM, between 0.6 μM and 0.7 μM, between 0.6 μM and 0.75 μM, between 0.65 μM and 0.7 μM, between 0.65 μM and 0.75 μM, between 0.65 μM and 0.8 μM, between 0.7 μM and 0.75 μM, between 0.7 μM and 0.8 μM, between 0.7 μM and 0.85 μM, between 0.75 μM and 0.8 μM, between 0.75 μM and 0.85 μM, between 0.75 μM and 0.9 μM, between 0.8 μM and 0.85 μM, between 0.8 μM and 0.9 μM, between 0.8 μM and 0.95 μM, between 0.85 μM and 0.9 μM, between 0.85 μM and 0.95 μM, between 0.85 μM and 1 μM, between 0.9 μM and 0.95 μM, between 0.9 μM and 1 μM, or between 0.95 μM and 1 μM

In embodiments, the therapeutically effective dose of CHIR99021 is between 1 μM and 20 μM. In embodiments, the therapeutically effective dose of CHIR99021 is between 1 μM and 5 μM, between 1 μM and 10 μM, between 1 μM and 15 μM, between 5 μM and 10 μM, between 5 μM and 15 μM, between 5 μM and 20 μM, between 10 μM and 15 μM, between 10 μM and 20 μM, or between 15 μM and 20 μM.

In embodiments, the therapeutically effective dose of DAPT is between 1 μM and 20 μM. In embodiments, the therapeutically effective dose of DAPT is between 1 μM and 5 μM, between 1 μM and 10 μM, between 1 μM and 15 μM, between 5 μM and 10 μM, between 5 μM and 15 μM, between 5 μM and 20 μM, between 10 μM and 15 μM, between 10 μM and 20 μM, or between 15 μM and 20 μM.

In embodiments, the therapeutically effective dose of SKL2001 is between 10 μM and 60 μM. In embodiments, the therapeutically effective dose of SKL2001 is between 10 μM and 20 μM, between 10 μM and 30 μM, between 10 μM and 40 μM, between 20 μM and 30 μM, between 20 μM and 40 μM, between 20 μM and 50 μM, between 30 μM and 40 μM, between 30 μM and 50 μM, between 30 μM and 60 μM, between 40 μM and 50 μM, between 40 μM and 60 μM, or between 50 μM and 60 μM.

In embodiments, the therapeutically effective dose is delivered to subject in need at least once daily or at least once weekly for at least two consecutive days or weeks. In embodiments, therapeutically effective dose is delivered to subject in need thereof at least once daily or at least once weekly for at least 3, 4, 5, 6, 7, 8, 9, 10, 11, 12, 13, 14, or 15 consecutive days or weeks. In embodiments, therapeutically effective dose is delivered to subject in need thereof at least once daily or at least once weekly for at least 1, 2, 3, 4, 5, 6, 7, 8, 9, 10, 11, or 12 consecutive weeks. In embodiments, therapeutically effective dose is delivered to subject in need thereof at least once daily or at least once weekly for at most 4, 5, 6, 7, 8, 9, 10, 11, 12, 13, 14, 15, 16, 17, 18, 19, or 20 consecutive days or weeks. In embodiments, therapeutically effective dose is delivered to subject in need thereof at least once daily or at least once weekly for at most 1, 2, 3, 4, 5, 6, 7, 8, 9, 10, 11, or 12 consecutive weeks or months. In embodiments, therapeutically effective dose is delivered to subject in need thereof is administered at least once for at least 1, 2, 3, 4, 5, 6, 7, 8, 9, 10, 11, or 12 consecutive months or years, chronically for a subject's entire life span, or an indefinite period of time. In embodiments, therapeutically effective dose is delivered to subject in need thereof once a year for 2 consecutive years, 3 consecutive years, or 5 consecutive years. In embodiments, therapeutically effective dose is delivered to subject in need thereof once a year for 2 consecutive years. In embodiments, therapeutically effective dose is delivered to subject in need thereof once a year for 3 consecutive years. In embodiments, therapeutically effective dose is delivered to subject in need thereof once a year for 5 consecutive years.

In embodiments, a composition herein, consists essentially of SKL2001. In embodiments, a composition herein consists essentially of SKL2001 and a second compound selected from the group consisting of SB431542, LDN193189, CHIR99021, and DAPT, or a combination thereof.

Compositions comprising one or more of the described compounds can be provided in pharmaceutical formulations. The form of pharmaceutical preparation is not particularly limited, but generally comprises the stated active ingredient(s) and at least one inactive ingredient. In embodiments suitable pharmaceutical compositions can be prepared by mixing any one or combination of the compounds with a pharmaceutically-acceptable carrier, diluent or excipient, and suitable such components are well known in the art. Some examples of such carriers, diluents and excipients can be found in: Remington: The Science and Practice of Pharmacy (2005) 21st Edition, Philadelphia, Pa. Lippincott Williams & Wilkins. In embodiments, the pharmaceutical formulations are suitable for delivering the active ingredients across the blood-brain barrier, and/or to the spinal cord or other components of the central nervous system. Such compositions can comprise, for example, lipid formulations or other nano-particle based delivery systems. In embodiments, the pharmaceutical formulation is suitable for oral administration, and thus can be provided in an aerosolized, liquid or solid dosage form. Solid dosage forms include but are not necessarily limited to tablets, capsules, caplets, and strips, for swallowing or oral dissolution, and may be provided for rapid or extended release, or to release distinct compounds in a desirable series over a period of time. Separate pharmaceutical compositions comprising one or any combination of the compounds can also be used. In embodiments, one or more of the described compounds are injected directly into a tumor, such as a glioma, or are administered directly to the brain and/or CAN. Thus, with respect to the administration of the pharmaceutical formulations, the route of administration can be any suitable route. In embodiments, the composition comprising the compound(s) is delivered orally. In other non-limiting embodiments, the composition is administered intravenously, parenterally, subcutaneously, intraperitoneally, transdermally, by intranasal instillation, by implantation, intraarterially, or by intrathecal administration. In embodiments, an implantable medical device can be used, such as a pump, including but not limited to an osmotic pump. In embodiments the compositions comprising the compounds is delivered via an intracranial route.

In embodiments, the therapeutically effective dose of this disclosure, achieves a remission, cure, response rate, or resolution rate of the glioma of at least about 50%. In embodiments, a therapeutically effective dose eliminates, reduces, slows, or delays, one or more of the glioma symptoms. Non-limiting examples of glioma symptoms include headache, seizures, nausea and vomiting, confusion, weakness, numbness, and imbalance. In embodiments, glioma symptom is a psychiatric symptom. Non-limiting examples of psychiatric symptoms include depression, irritability, sadness or apathy, social withdrawal, insomnia, fatigue, lack of energy, obsessive-compulsive disorder, mania, bipolar disorder, and weight loss.

In embodiments, therapeutically effective dose achieves remission, cure, response rate, or resolution rate of therapeutically effective dose of between about 10% and about 100% or more. In embodiments, therapeutically effective dose achieves remission, cure, response rate, or resolution rate of a glioma symptom between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, therapeutically effective dose eliminates, reduces, slows, or delays, one or more glioma symptoms between 10% and 100%, such as between 10% to about 15%, between 10% and 20%, between 10% and 25%, between 15% and 20%, between 15% and 25%, between 15% and 30%, between 20% and 25%, between 20% and 30%, between 20% and 35%, between 25 and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, a glioma symptom is assessed on the day of treatment, 1 day post treatment, 3 months post treatment, 6 months post treatment, 1 year post treatment and every year thereafter post treatment.

In embodiments, a glioma symptom is assessed between 1 day post treatment and 7 days post treatment. In embodiments, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In embodiments, symptoms can be assessed between 1 week post treatment and 4 weeks post treatment. In embodiments, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In embodiments, symptoms can be assessed between 1 month post treatment and 12 months post treatment. In embodiments, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In embodiments, symptoms can be assessed between 1 year post treatment and about 20 years post treatment. In embodiments symptoms can be assessed between 1 year post treatment and 5 years post treatment, between 1 year post treatment and 10 years post treatment , between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

As used herein, the term “survival rate” refers to a cohort of subjects in a treatment group still alive after a given period of time after diagnosis of a glioma.

In embodiments, therapeutically effective dose achieves increase survival rate of between about 10% and 100% or more. In embodiments, a therapeutically effective dose achieves an increase in survival rate of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

As used herein, the term “life expectancy” refers to a period of time a subject is expected to live. In embodiments, life expectancy is determined by gender. In embodiments, life expectancy is determined by genetics. In embodiments, life expectancy is determined by illness. In embodiments, life expectancy is determined by education. In embodiments, life expectancy is determined by mental health. In embodiments, life expectancy is determined by the population of a country.

In embodiments, therapeutically effective dose increases life expectancy of between about 10% and 100% or more. In embodiments, a therapeutically effective dose increases life expectancy of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, therapeutically effective dose reduces the amount of atrophy within the brain of a subject in need thereof between about 10% and 100% or more. In embodiments, a therapeutically effective dose reduces the amount of atrophy within the brain of a subject in need thereof between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, the amount of atrophy within the brain of a subject in need is assessed on the day of treatment, 1 day post treatment, 3 months post treatment, 6 months post treatment, 1 year post treatment and every year thereafter post treatment.

In embodiments, the amount of atrophy within the brain of a subject in need is assessed between 1 day post treatment and 7 days post treatment. In embodiments, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In embodiments, symptoms can be assessed between 1 week post treatment and 4 weeks post treatment. In embodiments, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In embodiments, symptoms can be assessed between 1 month post treatment and 12 months post treatment. In embodiments, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In embodiments, symptoms can be assessed between 1 year post treatment and about 20 years post treatment. In embodiments symptoms can be assessed between 1 year post treatment and 5 years post treatment, between 1 year post treatment and 10 years post treatment , between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

Non-limiting examples of tests to evaluate the amount of atrophy within the brain of a subject in need include Nissle staining, MRI, functional magnetic resonance fMRI, and PET scanning.

In embodiments, the disclosure includes an article of manufacture. In certain aspects, the article of manufacture includes a closed or sealed package that contains one or a combination of the compounds described herein. A combination of compounds and/or separate doses may be included in separate tablets, capsules or the like. The package can comprise one or more containers, such as closed or sealed vials, bottles, blister (bubble) packs, or any other suitable packaging for the sale, or distribution, or use of pharmaceutical agents. Thus, the package can contain pharmaceutical compositions which comprise SKL2001, and one SB431542, LDN193189, CHIR99021, and DAPT. For a combination of compounds, each compound can be provided separately in the same or distinct dosage formulations so that they can be delivered concurrently, or sequentially.

In addition to the pharmaceutical compositions, the package may contain printed information. The printed information can be provided on a label, or on a paper insert, or printed on the packaging material itself. The printed information can include information that identifies the active agents in the package, the amounts and types of inactive ingredients, an indication of what condition(s) the pharmaceutical composition(s) is intended to treat, and instructions for taking the pharmaceutical composition, such as the number of doses to take over a given period of time, the order to take the compositions, and the like. Thus, in various embodiments the disclosure includes a pharmaceutical composition of the invention packaged in a packaging material and identified in print, on or in the packaging material, that the composition is for use in the treatment any type of glioma. In embodiments, the printed material provides information about use of the pharmaceutical compositions for improving glioma symptoms. Non-limiting examples of glioma symptoms include headache, seizures, nausea and vomiting, confusion, weakness, numbness, and imbalance.

The following Example is provided to illustrate the invention, but is not intended to be limiting in any way.

### EXAMPLE

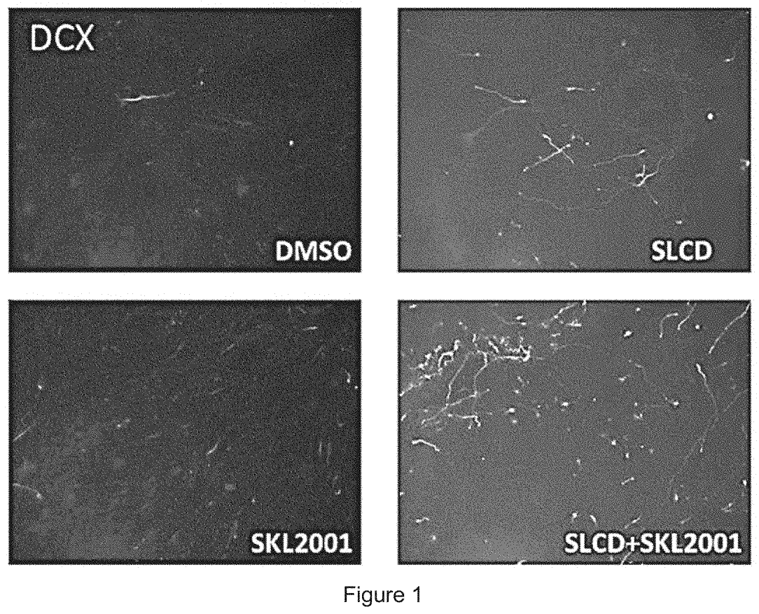

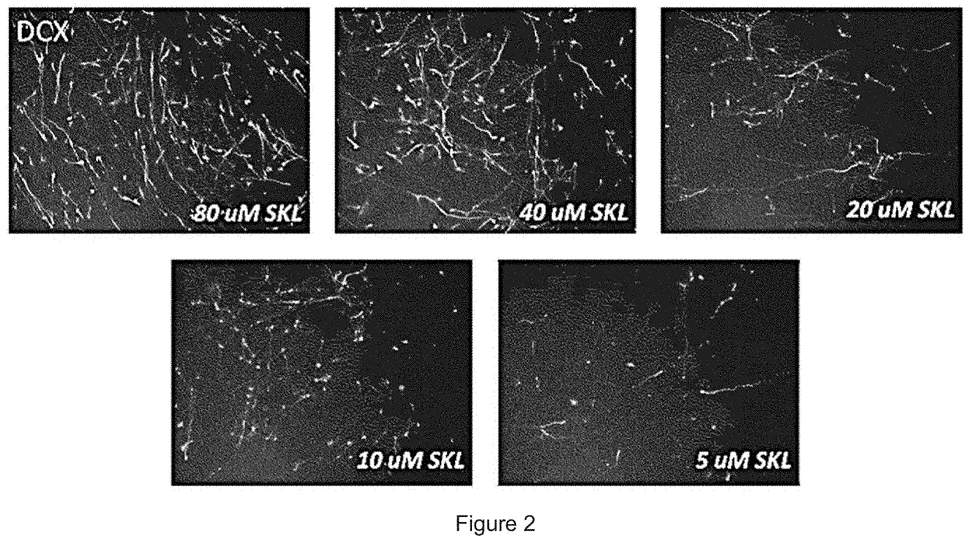

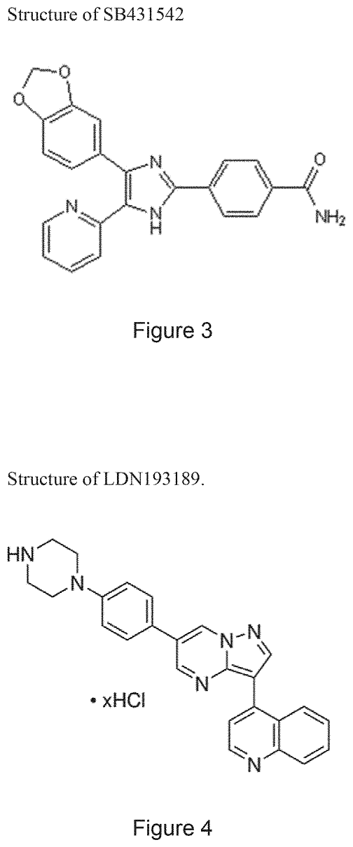

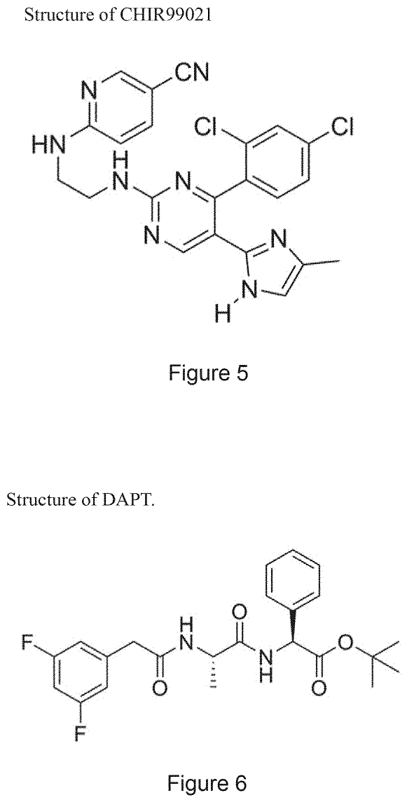

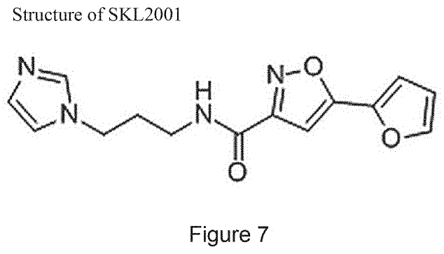

To produce the data depicted in FIG. 1 and FIG. 2, the following materials and methods were used.

Methods

U251 cells were maintained in minimal essential media (MEM) supplemented with 2 mM glutamine, 1% non-essential amino acids, 1 mM sodium pyruvate, and 10% fetal bovine serum. Fifty thousand cells were seeded on to coverslips into 24-well plates. After 4 days in MEM, the cells were treated with the compound cocktail or dimethyl sulfoxide for another 4 days in Ham's F-12 (F-12) media supplemented with N2, B27 and insulin. The five-compound cocktail, referred to as described above as SLCDS, contained 5 μM SB431542, 0.25 μM LDN193189, 5 μM CHIR99021, 5 μM DAPT, and 40 μM SKL2001. After SLCDS treatment, F-12 media was changed every 4 days for 12 days. A summary of the media used is shown in Table 1. Cells were then crosslinked with 4% paraformaldehyde for 12 minutes and washed with Tris buffered saline (TBS). After three 5-minute washes, cells were blocked with 2.5% bovine serum albumin in Tris-buffered saline (TBS) with 0.1% Triton X-100 (TBSTx) for 1 hour. Primary antibody (goat Doublecortin, 1:500 dilution, sc-8066, Santa Cruz) were incubated with fixed cells overnight at 4° C. After three 5-minute washes, the secondary antibody (anti-goat Cy5, 1:1000) was incubated with fixed cells overnight at 4° C. After three 5-minute washes, the cover slip was mounted using mounting media. The fluorescent signal was detected using an ECHO Revolve microscope.

TABLE 1
Summary of the media used for reprograming of U251 cells.

| | Days | | |
|---|---|---|---|
| Reagent | 1-4 | 5-8 | 9-20 |
| SLCDS | | X | |
| F-12 | | X | X |
| MEM | X | | |

Results

After U251 cells were allowed to grow to almost complete confluence treatment with SLCDS induced the expression of doublecortin in a SKL2001 dependent manner (FIG. 1). A dose-response relationship is established with SKL2001 in FIG. 2.

The following reference listing is not an indication that any of the references are material to patentability.
1 Cantrell, J. N. et al. Progress Toward Long-Term Survivors of Glioblastoma. Mayo Clinic Proceedings 94, 1278-1286, doi:10.1016/j.mayocp.2018.11.031 (2019).
2 Stupp, R. et al. Radiotherapy plus Concomitant and Adjuvant Temozolomide for Glioblastoma. New England Journal of Medicine 352, 987-996, doi:10.1056/NEJMoa043330 (2005).
3 Perry, J. R. et al. Short-Course Radiation plus Temozolomide in Elderly Patients with Glioblastoma. New England Journal of Medicine 376, 1027-1037, doi:10.1056/NEJMoa1611977 (2017).
4 Jackson, M., Hassiotou, F. & Nowak, A. Glioblastoma stem-like cells: at the root of tumor recurrence and a therapeutic target. Carcinogenesis 36, 177-185, doi:10.1093/carcin/bgu243 (2014).
5 Hombach-Klonisch, S. et al. Glioblastoma and chemoresistance to alkylating agents: Involvement of apoptosis, autophagy, and unfolded protein response. Pharmacology & Therapeutics 184, 13-41, doi: //doi.org/10.1016/j.pharmthera.2017.10.017 (2018).
6 Beier, D. et al. Temozolomide Preferentially Depletes Cancer Stem Cells in Glioblastoma. Cancer Research 68, 5706, doi:10.1158/0008-5472.CAN-07-6878 (2008).
7 William, D., Walther, M., Schneider, B., Linnebacher, M. & Classen, C. F. Temozolomide-induced increase of tumorigenicity can be diminished by targeting of mitochondria in in vitro models of patient individual glioblastoma. PLOS ONE 13, e0191511, doi:10.1371/journal.pone.0191511 (2018).
8 Gao, L. et al. Suppression of glioblastoma by a drug cocktail reprogramming tumor cells into neuronal like cells. Scientific Reports 9, 3462, doi:10.1038/s41598-019-39852-5 (2019).
9 Yuan, J. et al. Reprogramming glioblastoma multiforme cells into neurons by protein kinase inhibitors. Journal of Experimental & Clinical Cancer Research 37, 181, doi:10.1186/s13046-018-0857-5 (2018).
10 Rampazzo, E. et al. Wnt activation promotes neuronal differentiation of Glioblastoma. Cell Death & Disease 4, e500-e500, doi:10.1038/cddis.2013.32 (2013).
11 Yin, J.-C. et al. Chemical Conversion of Human Fetal Astrocytes into Neurons through Modulation of Multiple Signaling Pathways. Stem Cell Reports 12, 488-501, doi: //doi.org/10.1016/j.stemcr.2019.01.003 (2019).
12 Gwak, J. et al. Small molecule-based disruption of the Axin/β-catenin protein complex regulates mesenchymal stem cell differentiation. Cell Research 22, 237-247, doi:10.1038/cr.2011.127 (2012).

In an embodiment, the disclosure provides:

A method for converting glioma cells into neurons, the method comprising contacting the glioma cells with an effective amount of SKL2001. In an embodiment, the method further comprises contacting the glioma cells with at an effective amount of least one of SB431542, LDN193189, CHIR99021, or DAPT. In an embodiment, the glioma cells are contacted with a combination of SKL2001 and at least two of SB431542, LDN193189, CHIR99021, or DAPT. In an embodiment, the glioma cells are contacted with a combination of SKL2001 and at least three of SB431542, LDN193189, CHIR99021, or DAPT. In an embodiment, the glioma cells are contacted with a combination of SKL2001, SB431542, LDN193189, CHIR99021, and DAPT. In any of the foregoing embodiments, the neurons express doublecortin (DCX). In any of the foregoing embodiments, the glioma cells comprise glioblastoma cells. In an embodiment, the glioma cells are in a subject who has been diagnosed with a glioma. In an embodiment, the subject is a human. In an embodiment, the stage of glioma is selected from the group consisting of stage I, stage II, stage II, and stage IV. In an embodiment, the glioma cells are in subject who has been diagnosed with a astrocytoma. In an embodiment, the glioma cells are in a subject who has been diagnosed with glioblastoma. In any of the foregoing embodiments, the SKL2001 and at least one of the SB431542, LDN193189, CHIR99021, or the DAPT are the only active ingredients of a one or more pharmaceutical formulations with which the glioma cells are contacted. In an embodiment the disclosure provides a pharmaceutical composition comprising a combination of SKL2001 and at least one of SB431542, LDN193189, CHIR99021, or DAPT. In an embodiment, the pharmaceutical composition comprises the SKL2001 and at least two of the SB431542, the LDN193189, the CHIR99021, or the DAPT. In an embodiment, the pharmaceutical composition comprises the SKL2001 and at least three of the SB431542, the LDN193189, the CHIR99021, or the DAPT. In an embodiment, the pharmaceutical composition comprises the SKL2001, the SB431542, the LDN193189, the CHIR99021, and the DAPT. In an embodiment, the pharmaceutical composition comprises the SKL2001 and at least one of the SB431542, the LDN193189, the CHIR99021, or the DAPT are the only active ingredients it the pharmaceutical formulation. In an embodiment, the disclosure provides an article of manufacture comprising a pharmaceutical composition comprising SKL2001 and at least one of SB431542, LDN193189, CHIR99021, or DAPT, the article of manufacture further comprising printed material providing an indication that the pharmaceutical composition is for use in treating a glioma. In an embodiment, the article of manufacture comprises a pharmaceutical formulation in which the SKL2001 and at least one of SB431542, LDN193189, CHIR99021, or DAPT are the only active ingredients in the pharmaceutical formulation. In an embodiment, article of manufacture comprises the. In an embodiment, the article of manufacture comprises a pharmaceutical formulation in which the SKL2001 and at least two of SB431542, LDN193189, CHIR99021, or DAPT, are present in the pharmaceutical formulation. In an embodiment, the article of manufacture comprises a pharmaceutical formulation in which the SKL2001 and at least three of SB431542, LDN193189, CHIR99021, or DAPT, are present in the pharmaceutical formulation. In an embodiment, the article of manufacture comprises a pharmaceutical formulation in which the SKL2001, SB431542, LDN193189, CHIR99021, and DAPT, are present in the pharmaceutical formulation.

While the invention has been described through specific embodiments, routine modifications will be apparent to those skilled in the art and such modifications are intended to be within the scope of the present invention.

What is claimed is:

1. A method for converting glioma cells into neurons, the method comprising contacting the glioma cells with an effective amount of SKL2001.

2. The method of claim 1, further comprising contacting the glioma cells with at an effective amount of at least one of SB431542, LDN193189, CHIR99021, or DAPT.

3. The method of claim 2, wherein the glioma cells are contacted with a combination of SKL2001 and at least two of SB431542, LDN193189, CHIR99021, or DAPT.

4. The method of claim 2, wherein the glioma cells are contacted with a combination of SKL2001 and at least three of SB431542, LDN193189, CHIR99021, or DAPT.

5. The method of claim 2, wherein the glioma cells are contacted with a combination of SKL2001, SB431542, LDN193189, CHIR99021, and DAPT.

6. The method of any one of claims 1-5, wherein the neurons express doublecortin (DCX).

7. The method of any one of claims 1-5 wherein the glioma cells comprise glioblastoma cells.

8. The method of claim 6, wherein the glioma cells are in a subject who has been diagnosed with a glioma.

9. The method of claim 8, wherein the subject is a human.

10. The method of claim 8, wherein the stage of glioma is selected from the group consisting of stage I, stage II, stage II, and stage IV.

11. The method of claim 8, wherein the glioma cells are in subject who has been diagnosed with a astrocytoma.

12. The method of claim 11, wherein the glioma cells are in a subject who has been diagnosed with glioblastoma.

13. The method of any one of claims 1-5, wherein the SKL2001 and at least one of the SB431542, LDN193189, CHIR99021, or the DAPT are the only active ingredients of a one or more pharmaceutical formulations with which the glioma cells are contacted.

14. A pharmaceutical composition comprising a combination of SKL2001 and at least one of SB431542, LDN193189, CHIR99021, or DAPT.

15. The pharmaceutical composition of claim 14, comprising the SKL2001 and at least two of the SB431542, the LDN193189, the CHIR99021, or the DAPT.

16. The pharmaceutical composition of claim 14, comprising the SKL2001 and at least three of the SB431542, the LDN193189, the CHIR99021, or the DAPT.

17. The pharmaceutical composition of claim 14, comprising the SKL2001, the SB431542, the LDN193189, the CHIR99021, and the DAPT.

18. The pharmaceutical composition of claim 14, wherein the SKL2001 and at least one of the SB431542, the LDN193189, the CHIR99021, or the DAPT are the only active ingredients it the pharmaceutical formulation.

19. An article of manufacture comprising a pharmaceutical composition comprising SKL2001 and at least one of SB431542, LDN193189, CHIR99021, or DAPT, the article of manufacture further comprising printed material providing an indication that the pharmaceutical composition is for use in treating a glioma.

20. The article of manufacture of claim 19, wherein the SKL2001 and at least one of SB431542, LDN193189, CHIR99021, or DAPT are the only active ingredients in the pharmaceutical formulation.

21. The article of manufacture of claim 20, wherein the SKL2001 and at least two of SB431542, LDN193189, CHIR99021, or DAPT, are present in the pharmaceutical formulation.

22. The article of manufacture of claim 21, wherein the SKL2001 and at least three of SB431542, LDN193189, CHIR99021, or DAPT, are present in the pharmaceutical formulation.

23. The article of manufacture of claim 21, wherein the SKL2001, SB431542, LDN193189, CHIR99021, and DAPT, are present in the pharmaceutical formulation.
